(19) United States
(12) Patent Application Publication
(10) Pub. No.: US 2023/0210540 A1
(43) Pub. Date: Jul. 6, 2023

(54) INJECTOR DEVICES FOR DELIVERING MATERIAL TO VASCULAR DEFECTS AND ASSOCIATED SYSTEMS AND METHODS

(71) Applicant: Covidien LP, Mansfield, MA (US)

(72) Inventors: Adin Douglas Martineau, Santa Ana, CA (US); Ali M. Hussain, Mission Viejo, CA (US); John I. Muri, Laguna Niguel, CA (US); Junwei Li, Irvine, CA (US); Jared Akira Shimizu, Tustin, CA (US); Andyanhdzung Huynh, Westminster, CA (US); Robert A. Pecor, Aliso Viejo, CA (US); Aarya Cyril Mecwan, Saratoga, CA (US)

(21) Appl. No.: 18/147,994

(22) Filed: Dec. 29, 2022

Related U.S. Application Data
(60) Provisional application No. 63/266,351, filed on Jan. 3, 2022.
(60) Provisional application No. 63/269,764, filed on Mar. 22, 2022.

(51) Int. Cl. A61B 17/12

(52) U.S. Cl. CPC A61B 17/12186; A61B 17/12113; A61B 2017/1205

(57) ABSTRACT

Systems, methods, and devices for treating vascular defects are disclosed herein. In some embodiments, a method of treating an aneurysm includes positioning a distal portion of an elongated member near or within an aneurysm. The method can include introducing an embolic composition into a lumen of the elongated member using an injector device coupled to a proximal portion of the elongated member. The injector device can pressurize the embolic composition to a pressure of at least 10,000 psi. The method can also include delivering the embolic composition into the aneurysm via the elongated member.

### CROSS-REFERENCE TO RELATED APPLICATION(S)

The present application claims the benefit of priority to U.S. Provisional Patent Application No. 63/266,351, filed Jan. 3, 2022, and U.S. Provisional Patent Application No. 63/269,764, filed Mar. 22, 2022, each of which is incorporated by reference herein in its entirety.

### TECHNICAL FIELD

The present technology generally relates to medical devices, and in particular, to injector devices for delivering material to vascular defects and associated systems and methods.

### BACKGROUND

An intracranial aneurysm is a portion of an intracranial blood vessel that bulges outward from the blood vessel's main channel. This condition often occurs at a portion of a blood vessel that is abnormally weak because of a congenital anomaly, trauma, high blood pressure, or for another reason. Once an intracranial aneurysm forms, there is a significant risk that the aneurysm will eventually rupture and cause a medical emergency with a high risk of mortality due to hemorrhaging. When an unruptured intracranial aneurysm is detected or when a patient survives an initial rupture of an intracranial aneurysm, vascular surgery is often indicated. One conventional type of vascular surgery for treating an intracranial aneurysm includes using a microcatheter to dispose a platinum coil within an interior volume of the aneurysm. Over time, the presence of the coil should induce formation of a thrombus. Ideally, the aneurysm's neck closes at the site of the thrombus and is replaced with new endothelial tissue. Blood then bypasses the aneurysm, thereby reducing the risk of aneurysm rupture (or re-rupture) and associated hemorrhaging. Unfortunately, long-term recanalization (i.e., restoration of blood flow to the interior volume of the aneurysm) after this type of vascular surgery occurs in a number of cases, especially for intracranial aneurysms with relatively wide necks and/or relatively large interior volumes.

Another conventional type of vascular surgery for treating an intracranial aneurysm includes deploying a flow diverter within the associated intracranial blood vessel. The flow diverter is often a mesh tube that causes blood to preferentially flow along a main channel of the blood vessel while blood within the aneurysm stagnates. The stagnant blood within the aneurysm should eventually form a thrombus that leads to closure of the aneurysm's neck and to growth of new endothelial tissue, as with the platinum coil treatment. One significant drawback of flow diverters is that it may take weeks or months to form aneurysmal thrombus and significantly longer for the aneurysm neck to be covered with endothelial cells for full effect. This delay may be unacceptable when risk of aneurysm rupture (or re-rupture) is high. Moreover, flow diverters typically require antiplatelet therapy to prevent a thrombus from forming within the main channel of the blood vessel at the site of the flow diverter. Antiplatelet therapy may be contraindicated shortly after an initial aneurysm rupture has occurred because risk of re-rupture at this time is high and antiplatelet therapy tends to exacerbate intracranial hemorrhaging if re-rupture occurs. For these and other reasons, there is a need for innovation in the treatment of intracranial aneurysms. Given the severity of this condition, innovation in this field has immediate life-saving potential.

### SUMMARY

The present technology is illustrated, for example, according to various aspects described below. These various aspects are provided as examples and do not limit the subject technology.

In one aspect of the present technology, a method of treating an aneurysm is provided. The method can include positioning a distal portion of an elongated member near or within an aneurysm. The method can also include introducing an embolic composition into a lumen of the elongated member using an injector device coupled to a proximal portion of the elongated member. The injector device can pressurize the embolic composition to a pressure of at least 10,000 psi. The method can also include delivering the embolic composition into the aneurysm via the elongated member.

In some embodiments, the embolic composition comprises a storage modulus of at least 80 Pa at 37° C. within a linear viscoelastic range of the embolic composition. The method can further include loading the embolic composition into the injector device. The embolic composition can have the storage modulus of at least 80 Pa at 37° C. within the linear viscoelastic range of the embolic composition before being loaded into the injector device. The embolic composition can be delivered into the aneurysm at a rate of at least 0.2 milliliters per minute.

In some embodiments, the injector device includes an injector body coupled to a tip. The injector body and the tip can be made at least partially out of a metallic material. The tip can include a body having a distal opening, a proximal opening, and an internal lumen extending between the distal and proximal openings. At least a portion of the distal opening can have a diameter of less than 0.025 inches.

In some embodiments, the tip includes a first elongated shaft disposed within the internal lumen of the body that extends through the distal opening and a second elongated shaft disposed within the internal lumen of the body. The first elongated shaft can have an outer surface and the second elongated shaft can be fixed to the outer surface of the first elongated shaft and surround a first portion of the first elongated shaft. The second elongated shaft can be wider than the distal opening. In some embodiments, the tip includes an insert disposed within the internal lumen of the body around the first and second elongated shafts. Optionally, the tip can include a third elongated shaft coupled to the outer surface of the first elongated shaft so that the third elongated shaft surrounds a second portion of the first elongated shaft spaced apart from the first portion.

In some embodiments, the method further includes deploying a neck cover from the elongated member while the distal portion of the elongated member is positioned near or within the aneurysm cavity such that the neck cover self-expands to assume a first expanded state within the aneurysm cavity. In some embodiments, delivering the embolic composition into the aneurysm causes the neck cover to transform into a second expanded state. In the first expanded state, the neck cover can have a first interior volume, and, in the second expanded state, the neck cover can have a second interior volume less than the first interior volume. In some embodiments, the method further includes releasing the neck cover after the neck cover transforms into the second expanded state.

In some embodiments, the method further includes terminating delivery of the embolic composition into the aneurysm, and dissipating residual pressure within the injector device using a pressure release mechanism.

In another aspect of the present technology, a method of treating an aneurysm is provided. The method can include positioning a distal end of an elongated shaft in an aneurysm cavity, introducing an embolic composition into a lumen of the elongated shaft using an injector device, and delivering an embolic composition into the aneurysm cavity. The injector device can be coupled to a proximal portion of the elongated shaft. The embolic composition can have a storage modulus of at least 80 Pa at 37° C. within a linear viscoelastic range of the embolic composition and can be delivered into the aneurysm cavity at a rate of at least 0.2 milliliters per minute.

In some embodiments, the method further includes loading the embolic composition into the injector device. The embolic composition can have a storage modulus of at least 80 Pa at 37° C. within a linear viscoelastic range of the embolic composition before being loaded into the injector device. When delivering the embolic composition, the injector device can pressurize the embolic composition to a pressure of at least 10,000 psi.

In some embodiments, the injector device includes a tip that has a body with a distal aperture. The tip can also include a first elongated shaft having an outer surface with the first elongated shaft being disposed at least partially within the body and extending through the distal aperture. The tip can include a second elongated shaft disposed within the body and fixed to the outer surface of the first elongated shaft such that the first elongated shaft is received within the second elongated shaft along a portion of its length. The second elongated shaft can be wider than the distal aperture.

In some embodiments, the method further includes terminating delivery of the embolic composition into the aneurysm, and dissipating residual pressure within the injector device using a pressure release mechanism.

In a further aspect of the present technology, an injector device for delivering an embolic composition for treating an aneurysm is provided. The injector device can include an injector body including a barrel that can hold the embolic composition and a rod disposed partially within the barrel. The rod can be movable relative to the barrel to pressurize the embolic composition within the barrel to a pressure of at least 10,000 psi. The injector device can also include a tip coupled to the injector body. The tip can include a body comprising a cavity having a stop therein. The tip can also include a first elongated shaft disposed at least partially within the cavity and extending through the body. The first elongated shaft can form a lumen between the injector body and the tip. The tip can also include a second elongated shaft disposed within the cavity. The second elongated shaft can couple to the first elongated shaft and surround a portion of the first elongated shaft. The second elongated shaft can also abut the stop.

In some embodiments, the injector device further includes the embolic composition. The embolic composition can have a storage modulus of at least 80 Pa at 37° C. within a linear viscoelastic range of the embolic composition.

In some embodiments, the body of the tip includes a proximal aperture and a distal aperture, with the cavity extending between the proximal and distal apertures. The first elongated shaft can have a diameter of less than 0.025 inches and the second elongated shaft can include a diameter that is larger than the diameter of the distal aperture. The first elongated shaft can include an outer surface and the second elongated shaft can be welded to the outer surface of the first elongated shaft such that the first elongated shaft surrounds a first portion of the first elongated shaft. Optionally, the tip can include a third elongated shaft coupled to and surrounding a second portion of the first elongated shaft, with the third elongated shaft being spaced apart from the second elongated shaft. The first, second, and third elongated shafts can be concentric. The tip can also comprise a fourth elongated shaft. The first and third elongated shafts can be received within the fourth elongated shaft such that the fourth elongated shaft surrounds a portion of the first and third elongated shafts. In some embodiments, the injector body and the tip can be made at least partially out of a metallic material.

In some embodiments, the rod is coupled to the barrel through a connector assembly. The connector assembly can retain the positioning of the rod with respect to the barrel. The connector assembly can couple to the barrel at a flange, which can be included at the proximal end portion of the barrel. The connector assembly can include a first connector surrounding the proximal end portion of the barrel, a second connector coupled to the first connector such that the flange of the barrel is interposed between the first and second connectors, and a third connector received within the second connector. The third connector can be coupled to the rod.

In some embodiments, the injector device further includes a handle coupled to the rod. The handle can include a plurality of prongs.

In some embodiments, the injector device further includes a pushing member coupled to a distal end portion of the rod. The rod can be configured to apply a distal force to the pushing member, and the pushing member can be configured to expand radially outward when the distal force is applied. The injector device can further include a backing ring disposed around the rod proximal to the pushing member.

In some embodiments, the injector device further includes a pressure release mechanism coupled to the injector body. The pressure release mechanism can be movable between a first configuration configured to maintain the pressure on the embolic composition, and a second configuration configured to dissipate the pressure on the embolic composition. The pressure release mechanism can include a first connector coupled to the barrel and a second connector coupled to the rod. The second connector can include an upper connector portion and a lower connector portion collectively defining an aperture configured to receive the rod. When the pressure release mechanism is in the first configuration, the pressure release mechanism can prevent the rod from moving proximally relative to the barrel. When the pressure release mechanism is in the second configuration, the pressure release mechanism can permit the rod to move proximally relative to the barrel. When the pressure release mechanism is in the first configuration, the upper connector portion and the lower connector portion can be positioned proximate to each other such that the aperture has a first, smaller size configured to engage the rod. When the pressure release mechanism is in the second configuration, the upper connector portion and the lower connector portion can be spaced apart from each other such that the aperture has a second, larger size configured to disengage from the rod.

In a further aspect of the present technology, an injector device for delivering an embolic composition for treating an aneurysm is provided. The injector device can include an injector body that can contain the embolic composition. The injector body can pressurize the embolic composition to a pressure of at least 10,000 psi. The injector device can also include a tip coupled to the injector body. The tip can include a body, a first elongated shaft disposed at least partially within the body, and a second elongated shaft disposed at least partially within the body and coupled to the first elongated shaft. The first elongated shaft can be received within the second elongated shaft. The second elongated shaft can retain the first elongated shaft within the body.

In some embodiments, the injector device includes the embolic composition. The embolic composition can have a storage modulus of at least 80 Pa at 37° C. within a linear viscoelastic range of the embolic composition.

In some embodiments, the body of the tip includes a distal aperture, and the second elongated shaft has a diameter that is larger than a diameter of the distal aperture. The first elongated shaft can include a diameter of less than 0.025 inches. The first elongated shaft can also include an outer surface and the second elongated shaft can be welded to the outer surface of the first elongated shaft.

In some embodiments, the injector device further includes a pressure release mechanism coupled to the injector body. The pressure release mechanism can be movable between a first configuration configured to maintain the pressure on the embolic composition, and a second configuration configured to dissipate the pressure on the embolic composition.

Additional features and advantages of the present technology are described below, and in part will be apparent from the description, or may be learned by practice of the present technology. The advantages of the present technology will be realized and attained by the structure particularly pointed out in the written description and claims hereof as well as the appended drawings.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1A is a partially schematic view of a system for treating an aneurysm in accordance with embodiments of the present technology.

FIG. 1B is an enlarged cross-sectional view of a distal portion of the delivery system shown in FIG. 1A.

FIGS. 2A-2E show an example method of treating an aneurysm with the systems of the present technology.

FIG. 3A shows a perspective view of an injector device configured in accordance with embodiments of the present technology.

FIG. 3B is an exploded view of an injector body of the injector device of FIG. 3A.

FIG. 3C is a cross-sectional view of the injector body shown in FIG. 3A.

FIGS. 3D-3G are side cross-sectional views of a pushing member of the injector body of FIG. 3A.

FIG. 3H is a closeup cross-sectional view of a portion of the injector body shown in FIG. 3C.

FIG. 3I is a top view of a tip of the injector device of FIG. 3A.

FIG. 3J is a cross-sectional view of the tip of FIG. 3I.

FIG. 3K is a closeup cross-sectional view of a portion of the tip shown in FIG. 3J.

FIG. 4A is a perspective view of an injector device with a pressure release mechanism configured in accordance with embodiments of the present technology.

FIG. 4B is a perspective view of the pressure release mechanism of FIG. 4A.

FIG. 4C is a front cross-sectional view of the pressure release mechanism of FIG. 4A.

FIG. 5 is a flow diagram illustrating an example method of treating an aneurysm in accordance with embodiments of the present technology.

### DETAILED DESCRIPTION

The present technology relates to systems, methods, and devices for treating vascular defects such as aneurysms. In some embodiments, the methods described herein include delivering an embolic composition into the aneurysm sac. The embolic composition can provide a complete or nearly complete volumetric filling of the internal volume of an aneurysm, and/or a complete or nearly complete coverage of the neck of the aneurysm with new endothelial tissue. These features, among others, can lead to a lower recanalization rate than that of platinum coil treatments and faster aneurysm occlusion than that of flow diverters. Additionally, the embolic compositions can be configured to biodegrade over time and thereby have little or no long-term mass effect.

Conventional treatment methods typically use either a low viscosity embolic composition that solidifies (e.g., forms a gel) when exposed to physiological conditions within the aneurysm, or precursor materials that are mixed immediately before delivery to form the final embolic composition. However, the former approach may present challenges with long-term storage stability, while the latter approach introduces additional steps into the treatment procedure and may introduce timing complications (e.g., if the composition gels too quickly, it may clog the delivery catheter; if the composition gels too slowly, it may leak out of the aneurysm). Highly viscous embolic compositions (e.g., embolic compositions having a storage modulus of at least 80 Pa at 37° C. within a linear viscoelastic range of the embolic composition) that are ready for use off-the-shelf without any mixing of precursor materials can address these issues but may be difficult to deliver using conventional systems and devices. For example, conventional delivery systems may not be able to generate and/or withstand the pressures needed to move a highly viscous embolic composition through small-diameter catheters (e.g., microcatheters) used for accessing intracranial aneurysms.

The systems, devices, and methods for treating aneurysms described herein can overcome these and/or other issues with delivering embolic compositions. For example, a method for treating an aneurysm in accordance with some embodiments of the present technology includes positioning a distal portion of an elongated member near or within an aneurysm. The method can include introducing an embolic composition (e.g., an embolic composition having a storage modulus of at least 80 Pa at 37° C. within a linear viscoelastic range of the embolic composition) into a lumen of the elongated member using an injector device that is coupled to a proximal portion of the elongated member. The injector device can be configured to pressurize the embolic composition to a high pressure, such as a pressure of at least 10,000 psi. For example, the injector device can include an injector body coupled to a tip, with the tip including a plurality of elongated shafts that are coupled to each other to withstand high pressures, such as by welding or other high strength coupling methods. The injector body can also include a barrel and a rod coupled to each other by a connector assembly that prevents the rod from being displaced backwards by the high pressures within the barrel. The embolic composition can then be delivered into the aneurysm via the elongated member.

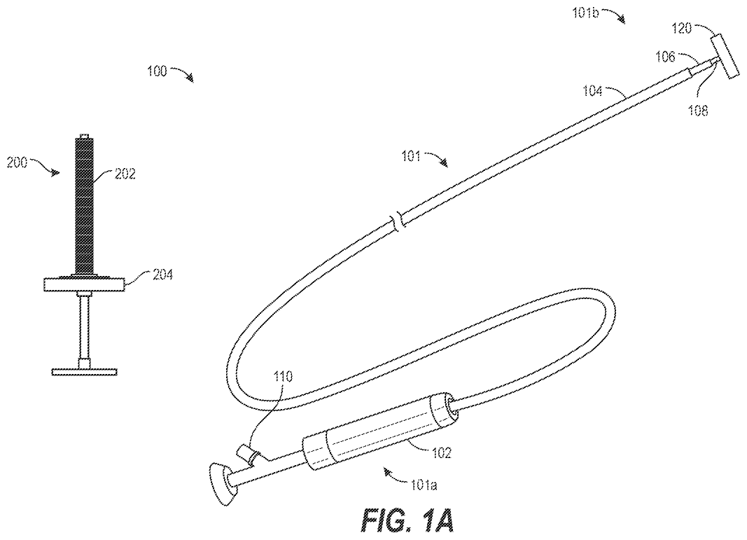

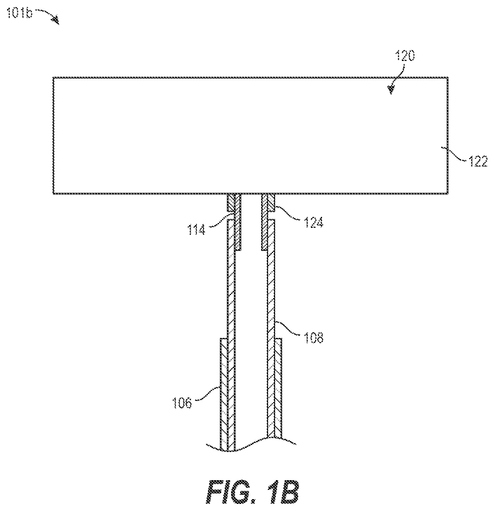

Specific details of systems, devices, and methods for treating aneurysms and/or other vascular defects in accordance with embodiments of the present technology are described herein with reference to FIGS. 1A-5. Although certain embodiments of these systems, devices, and methods may be described herein primarily or entirely in the context of treating saccular intracranial aneurysms, other contexts are within the scope of the present technology. For example, suitable features of described systems, devices, and methods for treating saccular intracranial aneurysms can be implemented in the context of treating non-saccular intracranial aneurysms, abdominal aortic aneurysms, thoracic aortic aneurysms, renal artery aneurysms, arteriovenous malformations, tumors (e.g. via occlusion of vessel(s) feeding a tumor), perivascular leaks, varicose veins (e.g. via occlusion of one or more truncal veins such as the great saphenous vein), hemorrhoids, and sealing endoleaks adjacent to artificial heart valves, covered stents, and abdominal aortic aneurysm devices, among other examples. Furthermore, it should be understood, in general, that other systems, devices, and methods in addition to those disclosed herein are within the scope of the present disclosure. For example, systems, devices, and methods in accordance with embodiments of the present technology can have different and/or additional configurations, components, procedures, etc. than those disclosed herein. Moreover, systems, devices, and methods in accordance with embodiments of the present disclosure can be without one or more of the configurations, components, procedures, etc. disclosed herein without deviating from the present technology.

The headings provided herein are for convenience only and do not interpret the scope or meaning of the claimed present technology. Embodiments under any one heading may be used in conjunction with embodiments under any other heading,

### I. Overview of Treatment Systems of the Present Technology

FIG. 1A shows a system 100 for treating aneurysms, such as cerebral aneurysms, according to one or more embodiments of the present technology. As shown in FIG. 1A, the system 100 comprises a delivery system 101, a neck cover 120, and an embolic kit 200. The neck cover 120 (shown schematically) is configured to be detachably coupled to the delivery system 101, and the delivery system 101 is configured to intravascularly position the neck cover 120 within an aneurysm. Representative examples of neck covers suitable for use with the system 100 are described in U.S. Pat. Nos. 8,142,456, 9,855,051, 10,327,781, U.S. Patent Application Publication No. 2020/0187953, U.S. Patent Application Publication No. 2021/0128169, and U.S. Patent Application Publication No. 2021/0153872, the disclosures of which are incorporated by reference herein in their entirety.

The embolic kit 200 comprises an embolic composition 202 and an injector device 204 (“injector 204”) configured to be fluidly coupled to a proximal portion of the delivery system 101 for injection of the embolic composition 202 into the aneurysm cavity. The embolic composition 202 can be delivered to a space between the neck cover 120 and the dome of the aneurysm to fill and occlude the aneurysm cavity. The neck cover 120 prevents migration of the embolic composition 202 into the parent vessel, and together the neck cover 120 and embolic composition 202 prevent blood from flowing into the aneurysm. As described in greater detail below, bioabsorption of the embolic composition 202 and endothelialization of the neck cover 120 cause the aneurysm wall to fully degrade, leaving behind a successfully remodeled (aneurysm free) region of the blood vessel.

As shown in FIG. 1A, the delivery system 101 has a proximal portion 101a configured to be extracorporeally positioned during treatment and a distal portion 101b configured to be intravascularly positioned at or within an aneurysm. The delivery system 101 may include a handle 102 at the proximal portion 101a and a plurality of elongated shafts extending between the handle 102 and the distal portion 101b. In some embodiments, for example as shown in FIG. 1A, the delivery system 101 may include a first elongated shaft 104 (such as a guide catheter or balloon guide catheter), a second elongated shaft 106 (such as a microcatheter) configured to be slidably disposed within a lumen of the first elongated shaft 104, and a third elongated shaft 108 configured to be slidably disposed within a lumen of the second elongated shaft 106. The delivery system 101 and/or the third elongated shaft 108 is configured to be detachably coupled at its distal end portion to the neck cover 120 via a connector 124 (see FIG. 1B) of the neck cover 120. In some embodiments, the delivery system 101 does not include the first elongated shaft 104.

The second elongated shaft 106 is generally constructed to track over a conventional guidewire in the cervical anatomy and into the cerebral vessels associated with the brain. The second elongated shaft 106 may also be chosen according to several standard designs that are generally available. For example, the second elongated shaft 106 can have a length that is at least 125 cm long, and more particularly may be between about 125 cm and about 175 cm long. The lumen of the second elongated shaft 106 is configured to slidably receive the neck cover 120 in a radially constrained state. The second elongated shaft 106 can have an inner diameter less than or equal to 0.006 inches (0.015 cm), 0.011 inches (0.028 cm), 0.015 inches (0.038 cm), 0.017 inches (0.043 cm), 0.021 inches (0.053 cm), or 0.027 inches (0.069 cm).

The third elongated shaft 108 can be movable within the first and/or second elongated shafts 104, 106 to position the neck cover 120 at a desired location. The third elongated shaft 108 can be sufficiently flexible to enable manipulation, e.g., advancement and/or retraction, of the neck cover 120 through tortuous passages. Tortuous passages can include, for example, catheter lumens, microcatheter lumens, blood vessels, urinary tracts, biliary tracts, and airways. The third elongated shaft 108 can be formed of any material and in any dimensions suitable for the task(s) for which the system 100 is to be employed. In some embodiments, at least the distal portion of the third elongated shaft 108 can comprise a flexible metal hypotube. The hypotube, for example, can be laser cut along all or a portion of its length to impart increased flexibility. In some embodiments, the third elongated shaft 108 can be surrounded over some or all of its length by a lubricious coating, such as polytetrafluoroethylene (PTFE).

Referring still to FIGS. 1A and 1B, the embolic composition 202 may be pre-loaded into the injector 204 (as shown), or at least some of the embolic composition 202 may be provided separately. The embolic composition 202 can be any material suitable for forming a solid or semi-solid structure (e.g., a hydrogel) that partially or completely occludes the interior cavity of the aneurysm. For example, the embolic composition 202 can include one or more polymers, such as a synthetic polymer (e.g., poly(glycolide), poly(lactide), poly(vinyl alcohol)), a biopolymer (e.g., chitosan, gelatin, silk, cellulose, alginate, hyaluronic acid), or a combination thereof. The embolic composition 202 can optionally include one or more components to facilitate gelation and/or enhance storage stability, such as cross-linking agents, stabilizers, thickeners, spacers, etc. Optionally, the embolic composition 202 can include a contrast agent to enable visualization (e.g., iohexol, iopromide, ioversol, iopamidol, iodixanol, ioxilan, iothalamate/meglumine, ioxaglate/meglumine, diatrizoate/meglumine). The embolic composition 202 can be biodegradable or non-biodegradable.

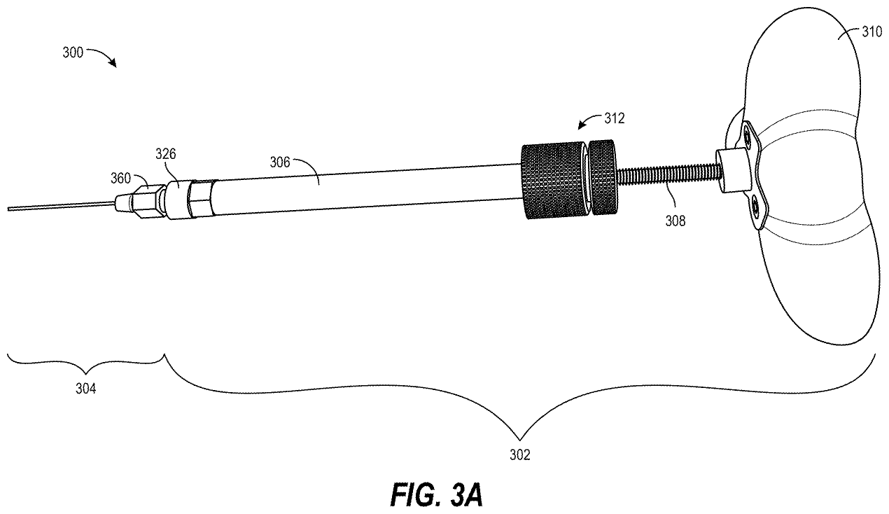

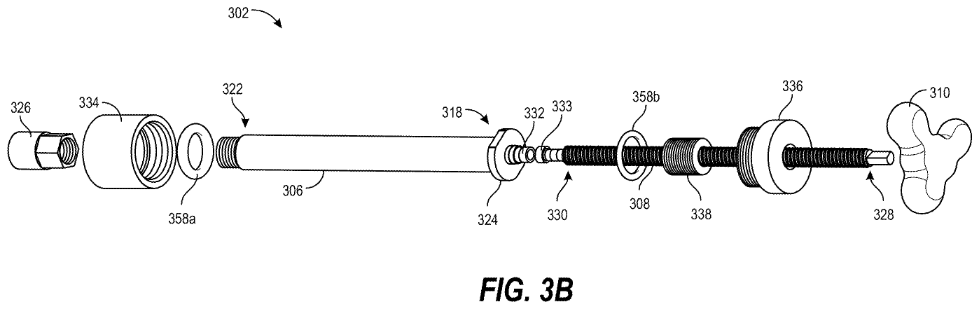

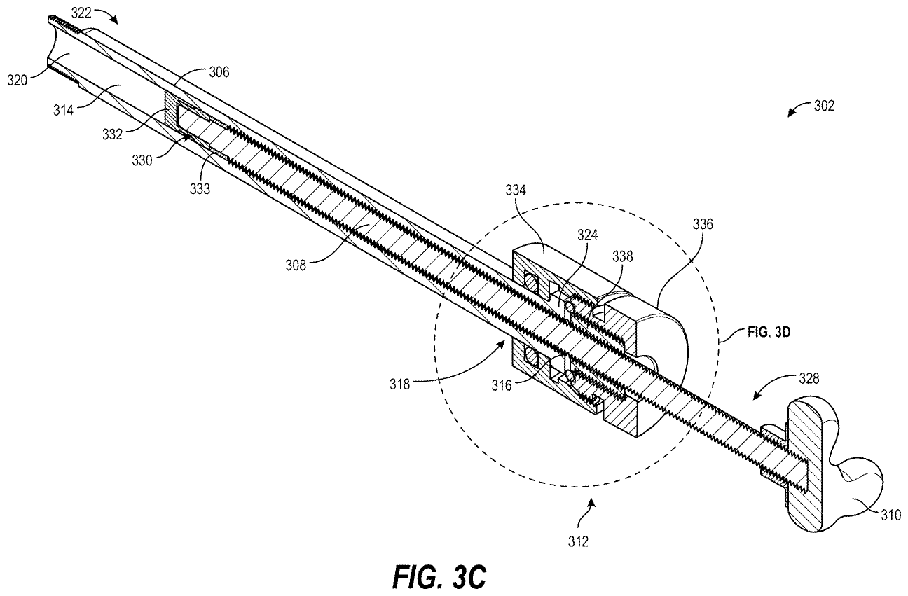

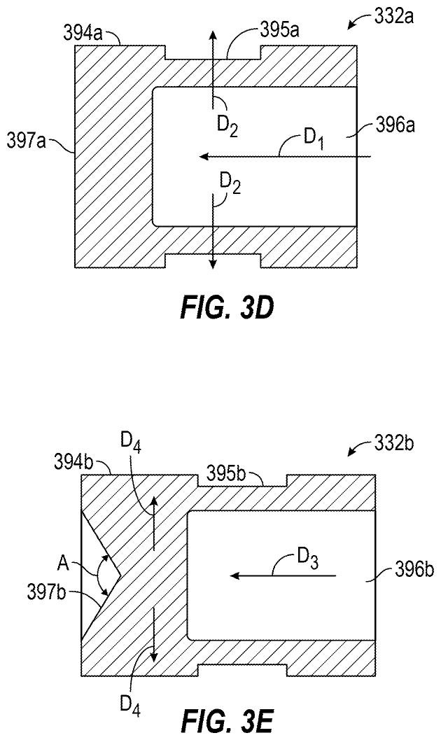

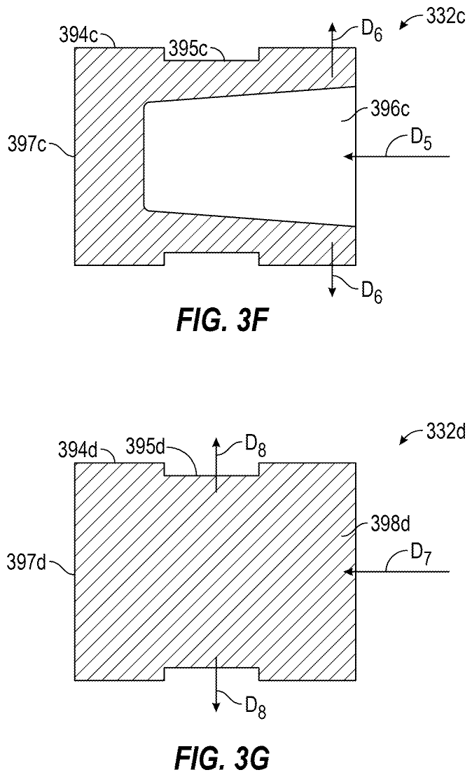

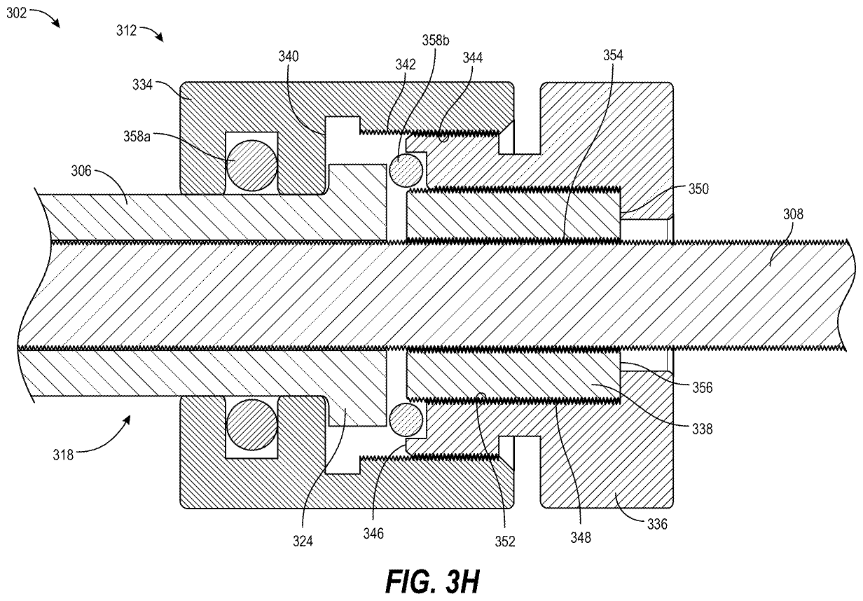

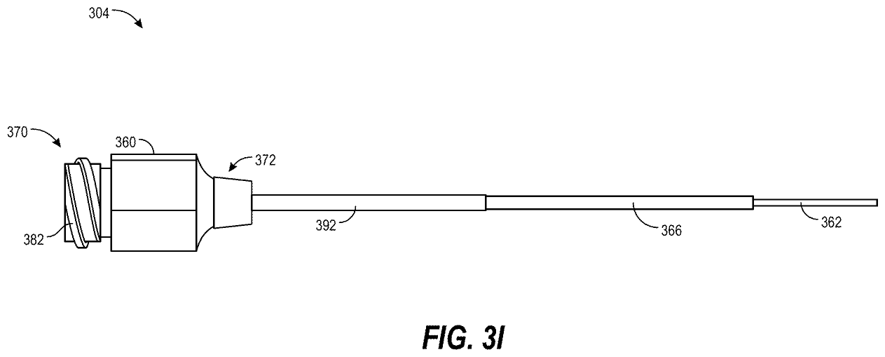

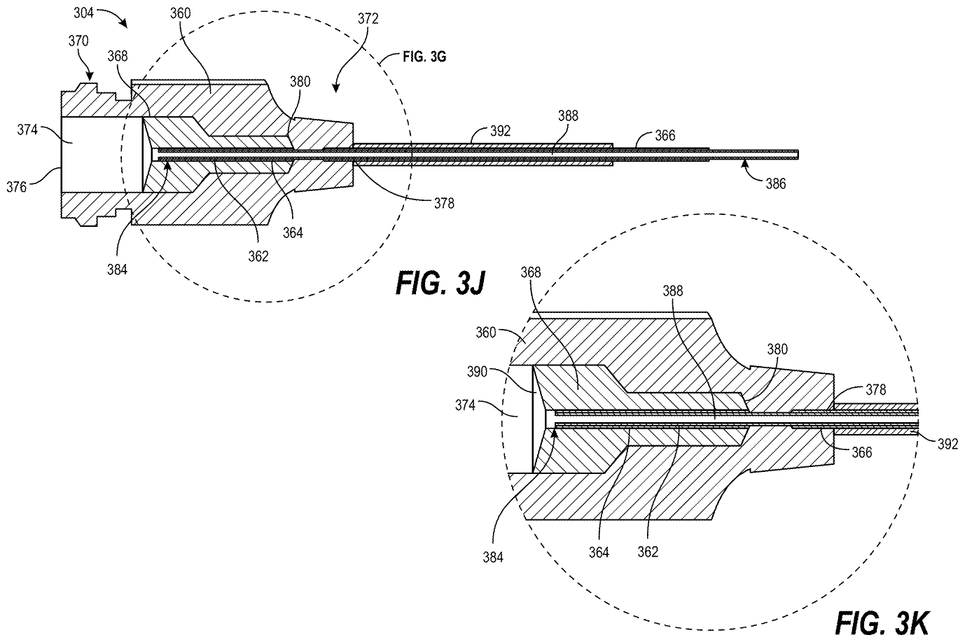

The embolic composition 202 can be provided in many different formats. In some embodiments, for example, the embolic composition 202 comprises two or more precursor materials that are mixed prior to or during delivery to the aneurysm. Upon mixing, the precursor materials can chemically react and/or physically interact to form a gel or other solid or semi-solid structure for occluding the aneurysm. Alternatively, the embolic composition 202 can be a preformed composition that is ready for use without any mixing of precursor materials. In such embodiments, the embolic composition 202 can be a highly viscous material that is sufficiently solid to fill and occlude the aneurysm without requiring further chemical reactions and/or physical interactions. The injector 204 can be configured to pressurize the embolic composition to a relatively high pressure (e.g., a pressure of at least 10,000 psi), as described in greater detail below with respect to FIGS. 3A-3K.

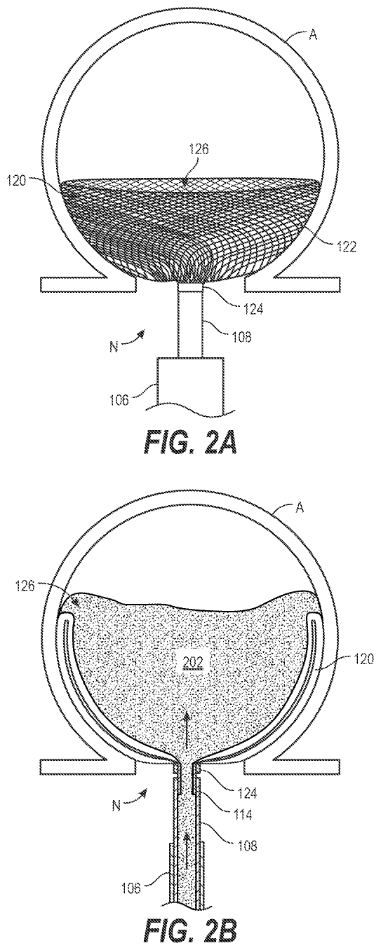

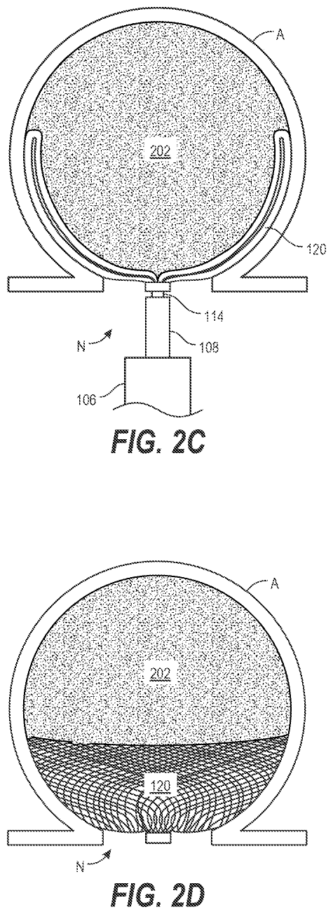

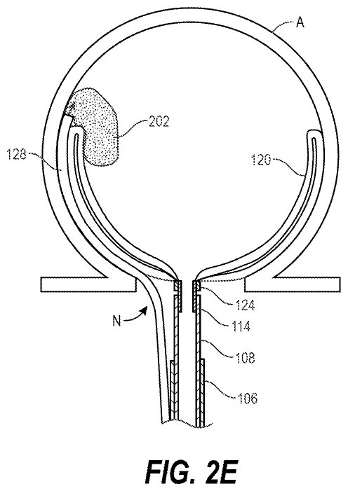

The system 100 can further include a conduit configured to guide the embolic composition 202 to a space between at least a portion of the neck cover 120 and the aneurysm dome. In some embodiments, the conduit is incorporated into the delivery system 101. For example, as depicted in the enlarged cross-sectional view of the distal portion 101b shown in FIG. 1B, the conduit can comprise a combination of the third elongated shaft 108 and an extension 114 fixed to a distal end portion of the third elongated shaft 108. The extension 114 can be a tubular member that extends distally from the third elongated shaft 108, through the connector 124, and through the neck cover 120, at least when the neck cover 120 is in an expanded state. When the neck cover 120 is collapsed within the lumen of the third elongated shaft 108 during delivery, a portion of the neck cover 120 may extend distally of the extension 114. The length of the extension 114 can be such that, when the distal portion 101b of the delivery system 101 is positioned at the aneurysm with the neck cover 120 in an expanded state (for example, as shown in FIG. 2A), a distal terminus of the extension 114 is even with the distal end of the connector 124, distal of the connector 124 but proximal of a distal end of the neck cover 120, or even with or distal of the distal end of the neck cover 120. It may be beneficial for the extension 114 to be as short as possible to ensure the extension 114 remains sufficiently spaced apart from the fragile aneurysm wall.

In some embodiments, the extension 114 comprises an atraumatic member, such as a soft, flexible coil. In other embodiments, the extension 114 comprises a flexible tube having a continuous sidewall (i.e., not formed of a coiled member). In any case, a distal end portion of the injector 204 can be fluidly coupled to a proximal end portion of the third elongated shaft 108 via a port 110. The port 110 can be located at the proximal portion 101a of the delivery system 101, such as on or proximal to the handle 102. The pressure generated at the injector 204 can cause the embolic composition 202 to flow through the lumen of the third elongated shaft 108, through the lumen of the extension 114, and into the aneurysm cavity. Once the embolic composition 202 has sufficiently filled the aneurysm cavity, the neck cover 120 and extension 114 can be detached via electrolytic detachment that severs a region of the extension 114 exposed between the third elongated shaft 108 and the neck cover 120.

According to several embodiments, the conduit may comprise an additional elongated shaft (not shown). The additional elongated shaft can be delivered to the aneurysm through one or more of the first, second, and/or third elongated shafts 104, 106, 108, or may be delivered separately (i.e., outside of) the delivery system 101. In such embodiments, a proximal end portion of the elongated shaft is configured to be fluidly coupled to the injector 204 via the port 110. Methods for delivering the embolic composition 202 through a separate elongated shaft are discussed below.

The neck cover 120 may comprise an expandable element having a low-profile or constrained state while positioned within a catheter (such as the second elongated shaft 106) for delivery to the aneurysm and an expanded, deployed state for positioning within the aneurysm. In some embodiments the neck cover 120 comprises a mesh 122 (shown schematically in FIG. 1B) and a connector 124 coupled to the mesh 122. The connector 124 is configured to be coupled to one or more components of the delivery system 101, such as the third elongated shaft 108 and/or extension 114. The mesh 122 can be formed of a resilient material and shape set such that upon exiting the second elongated shaft 106, the mesh 122 self-expands to a predetermined shape. The mesh 122 can have any shape or size in the expanded state that enables the mesh 122 to cover the aneurysm neck. In some embodiments, for example as shown in FIG. 2A, the mesh 122 can be configured to assume a bowl shape. Other shapes are possible. The mesh 122 has a porosity sufficient to prevent leakage of the embolic composition 202 into the parent vessel.

In some embodiments, the mesh 122 is formed of a plurality of braided filaments that have been heat-set to assume a predetermined shape when released from the constraints of the delivery catheter. The mesh 122 may be formed of metal wires, polymer wires, or both, and the wires may have shape memory and/or superelastic properties. The mesh 122 may be formed of 24, 32, 36, 48, 64, 72, 96, 128, or 144 filaments. The mesh 122 may be formed of a range of filament or wire sizes, such as wires having a diameter of from about 0.0004 inches to about 0.0020 inches, or of from about 0.0009 inches to about 0.0012 inches. In some embodiments, each of the wires or filaments have a diameter of about 0.0004 inches, about 0.0005 inches, about 0.0006 inches, about 0.0007 inches, about 0.0008 inches, about 0.0009 inches, about 0.001 inches, about 0.0011 inches, about 0.0012 inches, about 0.0013 inches, about 0.0014 inches, about 0.0015 inches, about 0.0016 inches, about 0.0017 inches, about 0.0018 inches, about 0.0019 inches, or about 0.0020 inches. In some embodiments, all of the filaments of the braided mesh 122 may have the same diameter. For example, in some embodiments, all of the filaments have a diameter of no more than 0.001 inches. In some embodiments, some of the filaments may have different cross-sectional diameters. For example, some of the filaments may have a slightly thicker diameter to impart additional strength to the braid. In some embodiments, some of the filaments can have a diameter of no more than 0.001 inches, and some of the filaments can have a diameter of greater than 0.001 inches. The thicker filaments may impart greater strength to the braid without significantly increasing the device delivery profile, with the thinner wires offering some strength while filling out the braid matrix density.

In some embodiments, the mesh 122 can be a non-braided structure, such as a laser-cut stent. Moreover, while the mesh 122 shown in FIGS. 2A-2D is a dual-layer mesh, in some embodiments the mesh 122 may comprise more or fewer layers (e.g., a single layer, three layers, four layers, etc.).

### II. Selected Methods for Treating Aneurysms with the Systems of the Present Technology

A physician may begin by intravascularly advancing the second elongated shaft 106 towards an intracranial aneurysm A with the neck cover 120 in a low-profile, collapsed state and coupled to a distal end portion of the third elongated shaft 108. A distal portion of the second elongated shaft 106 may be advanced through a neck N of the aneurysm A to locate a distal opening of the second elongated shaft 106 within an interior cavity of the aneurysm A. The third elongated shaft 108 may be advanced distally relative to the second elongated shaft 106 to push the neck cover 120 through the opening at the distal end of the second elongated shaft 106, thereby releasing the neck cover 120 from the shaft 108 and enabling the neck cover 120 to self-expand into an expanded, deployed state.

FIG. 2A shows the neck cover 120 in an expanded, deployed state, positioned in an aneurysm cavity and still coupled to the third elongated shaft 108. In the expanded, deployed state, the neck cover 120 may generally conform to the curved inner surface of the aneurysm A. In some embodiments the neck cover 120 assumes a predetermined shape that is concave towards the aneurysm dome and encloses an interior region 126.

As illustrated in FIG. 2B, the embolic composition 202 can be injected through the third elongated shaft 108 and extension 114 to a space between the neck cover 120 and an inner surface of the aneurysm wall. In other embodiments, the embolic composition 202 can be delivered through another elongated shaft (not shown) separate from the third elongated shaft 108 and extension 114. As additional embolic composition 202 is delivered, it fills the interior region 126 and all or a portion of the volume of the aneurysm cavity. It is beneficial to fill as much space in the aneurysm as possible, as leaving voids within the aneurysm sac may cause delayed healing and increased risk of aneurysm recanalization and/or rupture. While the scaffolding provided by the neck cover 120 across the neck helps thrombosis of blood form in any gaps and healing at the neck N, the substantial filling of the cavity prevents rupture acutely and does not rely on the neck cover 120. In some embodiments, the embolic composition 202 may fill greater than 40%, 50%, 60%, 65%, 70%, 75%, 80%, 85%, or 90% of the aneurysm sac volume.

FIG. 2C is a cross-sectional view of the neck cover 120 still attached to the delivery system just after completion of delivery of the embolic composition 202. During and after delivery, the embolic composition 202 exerts a substantially uniform downward pressure (i.e., towards the parent vessel) on the neck cover 120 that further seals and stabilizes the neck cover 120 around the neck N of the aneurysm A. Moreover, the embolic composition 202 along the distal wall 132 provides additional occlusion. In some embodiments, the embolic composition 202 completely or substantially completely occludes the pores of the adjacent layer or wall of the neck cover 120 such that blood cannot flow past the embolic composition 202 into the aneurysm cavity. It is desirable to occlude as much of the aneurysm as possible, as leaving voids of gaps can enable blood to flow in and/or pool, which may continue to stretch out the walls of aneurysm A. Dilation of the aneurysm A can lead to recanalization and/or herniation of the neck cover 120 and/or embolic composition 202 into the parent vessel and/or may cause the aneurysm A to rupture. Both conditions can be fatal to the patient.

As shown in FIG. 2D, once delivery of the embolic composition 202 is complete, the delivery system 101 and/or third elongated shaft 108 can be detached from the neck cover 120 (electrolytically or mechanically) and withdrawn from the patient's body. In those embodiments comprising a separate elongated shaft for delivering the embolic composition 202, the elongated shaft can be withdrawn before, during, or after detachment of the third elongated shaft 108 from the neck cover 120.

Over time, natural vascular remodeling mechanisms and/or bioabsorption of the embolic composition 202 may lead to formation of a thrombus and/or conversion of entrapped thrombus to fibrous tissue within the internal volume of the aneurysm A. These mechanisms also may lead to cell death at a wall of the aneurysm and growth of new endothelial cells between and over the filaments of the neck cover 120. Eventually, the thrombus and the cells at the wall of the aneurysm may fully degrade, leaving behind a successfully remodeled region of the blood vessel.

In some embodiments, contrast agent can be delivered during advancement of the neck cover 120 and/or embolic composition 202 in the vasculature, deployment of the neck cover 120 and/or embolic composition 202 at the aneurysm A, and/or after deployment of the neck cover 120 and/or embolic composition 202 prior to initiation of withdrawal of the delivery system. The contrast agent can be delivered through the second elongated shaft 106, the conduit, or through another catheter or device commonly used to deliver contrast agent. The aneurysm (and devices therein) may be imaged before, during, and/or after injection of the contrast agent, and the images may be compared to confirm a degree of occlusion of the aneurysm.

As shown in FIG. 2E, in some embodiments, the system 100 may comprise two separate elongated shafts (e.g., microcatheters), with one elongated shaft dedicated to delivery of the embolic composition 202 (e.g., a fourth elongated shaft 128), and the other elongated shaft dedicated to the delivery of the neck cover 120 (e.g., the third elongated shaft 108). In such embodiments, the fourth elongated shaft 128 can be fluidly coupled to the injector device 204 to form at least part of the conduit for conveying the embolic composition 202 into the aneurysm A. The fourth elongated shaft 128 may be intravascularly advanced to the aneurysm A and through the neck N such that that a distal tip of the fourth elongated shaft 128 is positioned within the aneurysm cavity. In some embodiments, the fourth elongated shaft 128 may be positioned within the aneurysm cavity such that the distal tip of the shaft 128 is near the dome of the aneurysm A.

The third elongated shaft 108 containing the neck cover 120 may be intravascularly advanced to the aneurysm A and positioned within the aneurysm cavity adjacent the fourth elongated shaft 128. The neck cover 120 may then be deployed within the aneurysm sac. As the neck cover 120 is deployed, it pushes the fourth elongated shaft 128 outwardly towards the side of the aneurysm A, and when fully deployed the neck cover 120 holds or “jails” the fourth elongated shaft 128 between an outer surface of the neck cover 120 and the inner surface of the aneurysm wall.

The embolic composition 202 may then be delivered through the fourth elongated shaft 128 to a position between the inner surface of the aneurysm wall and the outer surface of the neck cover 120. For this reason, it may be beneficial to initially position the distal tip of the fourth elongated shaft 128 near the dome (or more distal surface) of the aneurysm wall. This way, the “jailed” fourth elongated shaft 128 will be secured by the neck cover 120 such that the embolic composition 202 gradually fills the open space in the aneurysm sac between the dome and the neck cover 120.

### III. Selected Embodiments of Injector Devices and Associated Methods

FIGS. 3A-3K illustrate an injector device 300 configured in accordance with embodiments of the present technology. Referring first to FIG. 3A, which shows a perspective view of the injector device 300, the injector device 300 can be a syringe or similar device that is used to introduce an embolic composition into a treatment system to treat an aneurysm. For example, the injector device 300 can serve as the injector 204 of the system 100 of FIGS. 1A-2D and can be used to load the embolic composition 202 into the delivery system 100. The injector device 300 can couple to a proximal portion of the treatment system (e.g., to the port 110 of the delivery system 101 shown in FIG. 1A) and can include an injector body 302 coupled to a tip 304. As will be described in more detail below, the tip 304 can couple the injector device 300 to the treatment system to convey the composition from the injector body 302 to the treatment system. The tip 304 can provide a lumen that fluidly couples the injector body 302 to the conduit of the treatment system. The tip 304 can also withstand the very high pressure generated by the injector body 302.

In some embodiments, the injector body 302 generates and withstands a sufficient pressure to introduce the embolic composition into the treatment system and deliver the composition to an aneurysm within a patient. In some embodiments, for example, the injector body 302 generates a relatively high pressure compared to conventional delivery devices, such as a pressure of at least 4,000 psi, 5,000 psi, 6,000 psi, 7,000 psi, 8000, psi, 9,000 psi, 10,000 psi, 11,000 psi, 12,000 psi, 13,000 psi, 14,000 psi, 15,000 psi, or higher. The pressure produced by the injector body 302 may vary based on the viscosity of the embolic composition. For example, these high pressures can be advantageous for delivering a highly viscous embolic composition to the aneurysm, such as an embolic composition having a viscosity (e.g., dynamic viscosity at 20° C.) of at least 50 Pa-s, 100 Pa-s, 200 Pa-s, 500 Pa-s, or 1000 Pa-s. Storage modulus may be used as a proxy for viscosity in situations where the viscosity of the embolic composition is too high to be measured by conventional techniques. For example, the storage modulus of the embolic composition within the linear viscoelastic region can be at least 50 Pa, 80 Pa, 100 Pa, 200 Pa, 300 Pa, 400 Pa, 500 Pa, or 600 Pa when measured at 37° C. The linear viscoelastic region can correspond to no more than 20%, 15%, or 10% displacement of the embolic composition. The storage modulus of the embolic composition can be measured using techniques known to those of skill in the art, such as using a 40 mm 2° cone and plate rheometer (e.g., a TA Instruments Discovery HR 20 rheometer) oscillating at a suitable frequency (e.g., at or near 1 Hz).

The pressure used may also vary based on the inner diameter and/or length of the components used to deliver the embolic composition into the aneurysm. For example, the inner diameter of the conduit for delivering the embolic composition (e.g., the third elongated shaft 108 of FIGS. 1A and 1B) can be less than or equal to 0.02 inches, 0.015 inches, 0.014 inches, 0.013 inches, 0.012 inches, 0.011 inches, or 0.01 inches. The length of the conduit can be greater than or equal to 100 cm, 150 cm, 160 cm, 170 cm, 180 cm, 190 cm, or 200 cm.

FIG. 3B is an exploded view of the injector body 302, FIG. 3C is a cross-sectional view of the injector body 302, FIGS. 3D-3G are side cross-sectional views of pushing members 332a—d of the injector body 302, and FIG. 3H is a closeup cross-sectional view of a portion of the injector body 302. Referring first to FIGS. 3B and 3C together, the injector body 302 is configured to contain and deliver an embolic composition (e.g., the embolic composition 202 of FIGS. 1A-2D). As best seen in FIG. 3C, the injector body 302 includes a barrel 306 configured to hold the embolic composition, a rod 308 coupled to the barrel 306, a handle 310 coupled to the rod 308, and a connector assembly 312 coupled to the barrel 306 and the rod 308. At least a portion of the rod 308 is disposed within the barrel 306 and can be adjusted (e.g., translated and/or rotated) within the barrel 306 via the handle 310 to pressurize the embolic composition and push the embolic composition out of the barrel 306. The connector assembly 312 is configured to hold the rod 308 at a desired position with respect to the barrel 306 to prevent the pressurized embolic composition from displacing the rod 308.

As illustrated in FIG. 3C, the barrel 306 of the injector body 302 is an elongated, hollow structure (e.g., a generally cylindrical tube) extending from a proximal end portion 318 to a distal end portion 322. The barrel 306 can include an inner chamber 314 configured to hold the embolic composition. The inner chamber 314 can be a cavity, lumen, space, etc., within the barrel 306 extending from a first opening 316 at the proximal end portion 318 of the barrel 306 to a second opening 320 at the distal end portion 322 of the barrel 306. The inner chamber 314 can have any suitable set of dimensions, such as a volume of at least 0.5 mL, 1 mL, 1.2 mL, 1.5 mL, or 2 mL and/or a diameter of at least 0.15 inches, 0.16 inches, 0.165 inches, 0.168 inches, 0.17 inches, or 0.175 inches. The distal end portion 322 of the barrel 306 can be configured to couple to the tip 304. For example, the distal end portion 322 can include threading and/or other features for connecting to a coupling portion 326 (e.g., a Luer lock connector—FIG. 3B) which attaches to the tip 304, as described in greater detail below.

The rod 308 is an elongated shaft (e.g., a plunger or piston) having a proximal end portion 328 and a distal end portion 330. The proximal end portion 328 of the rod 308 can be positioned proximal to the barrel 306 while the distal end portion 330 of the rod 308 can be positioned within the inner chamber 314 of the barrel 306. As previously noted, the rod 308 can be configured to move (e.g., translate and/or rotate) within the barrel 306 to pressurize the inner chamber 314 of the injector body 302 and to deliver the embolic composition. For example, the rod 308 can translate along the longitudinal axis of the barrel 306 between the first and second openings 316, 320. In some embodiments, translating the rod 308 distally away from the first opening 316 and toward the second opening 320 reduces the volume of the inner chamber 314, which increases the pressure on the embolic composition held within the barrel 306. Conversely, translating the rod 308 proximally away from the second opening 320 and toward the first opening 316 increases the volume of the inner chamber 314.

In some embodiments, a pushing member 332 (e.g., a plunger tip) is coupled to or integrally formed with the rod 308 at the distal end portion 330 of the rod 308. The pushing member 332 can be configured to form a tight seal with the inner chamber 314 so that the rod 308 can more effectively pressurize the embolic composition within the barrel 306. For example, the pushing member 332 can have an inner diameter less than or equal to 0.168 inches and/or an outer diameter greater than or equal to 0.169 inches, 0.171 inches, or 0.173 inches. The pushing member 332 can be made partially or entirely out of a material that can withstand high pressures, such as a polymeric material (e.g., PTFE, polyether ether ketone (PEEK)). Optionally, the pushing member 332 can be made of a flexible and/or deformable material such that when force is applied to the pushing member 332, the pushing member 332 can expand radially outward to seal against the inner wall of the barrel 306, which can be advantageous for withstanding high pressures and/or preventing the embolic composition from flowing proximally within the barrel 306. As best seen in FIG. 3C, the injector body 302 can optionally include a backing ring 333 positioned around the rod 308 at a location proximal to the pushing member 332. The backing ring 333 can support the pushing member 332 and/or prevent the pushing member 332 from being displaced and/or deformed proximally due to high pressures within the barrel 306. In some embodiments, the backing ring 333 is made of a relatively stiff material that resists deformation under high pressures, such as a metal (e.g., stainless steel).

FIGS. 3D-3G illustrate various designs that can be used for the pushing member 332. For example, FIG. 3D shows a pushing member 332a having a generally cylindrical body 394a that is sized to press against the walls of the inner chamber 314. The outer surface of the body 394a can include a recess 395a (e.g., an annular indentation) near the central region of the body 394a. The body 394a includes an inner cavity 396a that receives the distal end portion 330 of the rod 308. In the illustrated embodiment, for example, the inner cavity 396a has a cylindrical shape that matches the cylindrical shape of the distal end portion 330. The pushing member 332a also includes a substantially flat distal face 397a that can press against the embolic composition within the inner chamber 314. In some embodiments, when the rod 308 pushes distally against the pushing member 332a (e.g., as indicated by arrow D₁), the central region of the body 394a adjacent or near the recess 395a deforms and expands radially outward (as indicated by arrows D₂) to seal against the inner walls of the barrel 306.

FIG. 3E shows another pushing member 332b. The components of the pushing member 332b (e.g., body 394b, recess 395b, inner cavity 396b) can be identical or generally similar to the corresponding components of the pushing member 332a of FIG. 3D, except that the pushing member 332b includes a concave distal face 397b. In the illustrated embodiment, the distal face 397b includes a conical recess, such that the central portion of the distal face 397b is indented relative to the peripheral portions of the distal face 397b. The conical recess can have any suitable apex angle A, such an angle A greater than or equal to 90°, 100°, 110°, 115°, 120°, 125°, 130°, 140°, or 150°. The conical recess can be shaped such that when the rod 308 pushes distally against the pushing member 332b (e.g., as indicated by arrow D₃), the distal region of the body 394b deforms and expands radially outward (as indicated by arrows D₄) to seal against the inner walls of the barrel 306.

FIG. 3F shows another pushing member 332c. The components of the pushing member 332c (e.g., body 394c, recess 395c, distal face 397c) can be identical or generally similar to the corresponding components of the pushing member 332a of FIG. 3D, except that the pushing member 332c includes a tapered inner cavity 396c, with the proximal end of the inner cavity 396c being wider than the distal end of the inner cavity 396c. In such embodiments, the distal end portion 330 of the rod 308 can also be tapered to conform to the shape of the inner cavity 396c. When the rod 308 pushes distally against the pushing member 332c (e.g., as indicated by arrow D₅), the proximal region of the body 394c can deform and expand radially outward (as indicated by arrows D₆) to seal against the inner walls of the barrel 306.

FIG. 3G shows another pushing member 332d. The components of the pushing member 332d (e.g., body 394d, recess 395d, distal face 397d) can be identical or generally similar to the corresponding components of the pushing member 332a of FIG. 3D, except that the pushing member 332d is a solid structure that does not include an inner cavity. In such embodiments, the rod 308 can contact and apply force to a proximal face 398d of the body 394d. When the rod 308 pushes distally against the pushing member 332d (e.g., as indicated by arrow D₇), the central region of the body 394d adjacent or near the recess 395d can deform and expand radially outward (as indicated by arrows D₈) to seal against the inner walls of the barrel 306.

Referring again to FIG. 3A, the injector body 302 can include a handle 310 coupled to the proximal end portion 328 of the rod 308 (the handle 310 is not shown to scale in FIGS. 3B and 3C). The handle 310 can be shaped to be grasped and manipulated by an operator (e.g., a physician or surgeon). For example, as best seen in FIG. 3B, the handle 310 can include an ergonomic body (e.g., a knob) having a multi-pronged (e.g., three-pronged) shape, which enables the operator to hold the handle 310 with their hand and maneuver the handle 310 as desired. The prongs of the handle 310 can provide a larger outer diameter that enables the operator to generate a higher torque with less force, while the inner diameter of the handle 310 can be smaller to accommodate multiple hand sizes. The prongs of the handle 310 can be spaced apart from each other so the operator can position their fingers between the prongs, which can make the handle 310 easier to grip and rotate. In other embodiments, however, the handle 310 can be shaped in a different manner from what is illustrated in FIGS. 3A-3C. For example, the handle 310 can include a different number of prongs (e.g., two, four, five, or more), the prongs can be shaped differently, or the handle 310 can have a different shape altogether (e.g., a square, circular, or triangular shape).

As previously noted, the handle 310 can be configured to adjust the positioning of the rod 308 with respect to the barrel 306. In some embodiments, the operator can push and/or pull the handle, which in turn pushes and/or pulls the rod 308 so the rod 308 translates within the barrel 306. Alternatively or in combination, the operator can move the rod 308 within the barrel 306 by rotating the handle 310. For example, the rod 308 can be threaded along a portion or the entirety of its length (e.g., between the proximal and distal end portions 328, 330; or near the distal end portion 330 only), which enables the rod 308 to translate within the barrel 306 when the operator rotates the handle 310. In such embodiments, each revolution of the rod 308 and handle 310 can cause a predetermined volume of the embolic composition to be delivered from the injector body 302, thus providing precise control over filling of the aneurysm cavity. For example, a single revolution of the rod 308 and handle 310 can cause no more than 50 μL, 20 μL, 10 μL, 5 μL, 1 μL 500 nL, 100 nL, 50 nL, 20 nL, 15 nL, 10 nL, 5 nL, 2 nL, or 1 nL of the embolic composition to be delivered from the injector body 302 and into the aneurysm.

The connector assembly 312 is configured to couple the barrel 306 and the rod 308 together and to hold the rod 308 at a desired position with respect to the barrel 306. As illustrated in FIGS. 3C and 3H, the connector assembly 312 includes a plurality of interconnected components, such as a first connector 334, a second connector 336 proximal to the first connector 334, and a third connector 338 received within to the second connector 336. As best seen in FIG. 3H, the first connector 334 has a generally tubular (e.g., cylindrical) body having a lumen that receives and surrounds the proximal end portion 318 of the barrel 306. In some embodiments, the first connector 334 surrounds a flange 324 that is formed at or near the proximal end portion 318 of the barrel 306. The first connector 334 can include a face 340 that is located near a central portion of the body within the lumen and is oriented proximally. The face 340 can be configured to contact a distal facing portion of the flange 324 and can act as a stop so the barrel 306 cannot move proximally relative to the first connector 334.

The second connector 336 is positioned proximal to the barrel 306 and at least a portion of the first connector 334. The second connector 336 includes a generally tubular (e.g., cylindrical) body having a lumen that receives and surrounds a portion of the rod 308. The second connector 336 can fit partially within the first connector 334 and can couple to the first connector 334 through one or more threaded portions. For example, the first connector 334 can include a threaded portion 342 on an inner surface at or near a proximal end of the first connector 334, and the second connector 336 can include a first threaded portion 344 on an outer surface at or near a proximal end of the second connector 336. The threaded portion 342 of the first connector 334 can mate with the first threaded portion 344 of the second connector 336, thus securing the second connector 336 to the first connector 334. The second connector 336 can also include a distally oriented face 346 at the distal end of the second connector 336. When the first and second connectors 334, 336 are coupled together, the flange 324 of the barrel 306 can be interposed and interlocked between the face 340 of the first connector 334 and the face 346 of the second connector 336. This arrangement can secure the barrel 306 to the connector assembly 312.

The third connector 338 is disposed within the lumen of the second connector 336 and includes a generally tubular (e.g., cylindrical) body including a lumen that receives and surrounds a portion of the rod 308. The outer surface of the third connector 338 can optionally include a first threaded portion 352 that mates with a second threaded portion 348 on an inner surface of the second connector 336, thus securing the third connector 338 within the lumen of the second connector 336. Alternatively or in combination, the third connector 338 can be coupled to the second connector 336 via a pin (e.g., an axially-oriented pin) or other fastener that prevents rotation of the third connector 338 relative to the second connector 336. Additionally, the proximal portion of the third connector 338 can include a proximally oriented face 356 that contacts a distally oriented face 350 at the proximal portion of the lumen of the second connector 336. The face 350 of the second connector 336 can act as a stop to prevent the third connector 338 from moving proximally relative to the second connector 336. The inner surface of the third connector 338 can include a second threaded portion 354 that extends along the length of the third connector 338. The second threaded portion 354 can mate with the threading on the rod 308 to secure the rod 308 within the lumen of the third connector 338.

In some embodiments, the connector assembly 312 includes one or more sealers (e.g., O-rings, gaskets, compressible inserts, etc.) positioned between the first, second, and/or third connectors 334,336,338, and/or other components of the injector body 302. For example, a first sealer 358a can be positioned between the first connector 334 and the barrel 306, and/or a second sealer 358b can be positioned between the second connector 336 and the flange 324. In some embodiments, the first sealer 358a secures the first connector 334 in a concentric disposition around the barrel 306, while the second sealer 358b serves as a bumper to secure the flange 324 to the connector assembly 312. In other embodiments, the first and/or second sealers 358a, 358b are optional and can be omitted.

The connector assembly 312 enables the rod 308 to move relative to the barrel 306 when actuated by an operator. For example, when the operator rotates the handle 310, the threaded coupling between the rod 308 and the third connector 338 enables the rod 308 to rotate relative to the third connector 338 (and thus, the connector assembly 312). Accordingly, the rod 308 can translate distally or proximally relative to the barrel 306 and connector assembly 312, depending on the direction of rotation. As previously discussed, the distal movement of the rod 308 can increase the pressure on the embolic composition within the barrel 306. When the operator releases the handle 310, the connector assembly 312 holds the barrel 306 and rod 308 in position and prevents the rod 308 from slipping proximally relative to the barrel 306 due to the pressurized embolic composition. Specifically, the rod 308 can be secured to the third connector 338 by the threaded coupling between these components, the third connector 338 can be secured to the second connector 336 by the threaded coupling between these components and by the face 350 acting as a stop, the second connector 336 can be secured to the first connector 334 by the threaded coupling between these components, and the first connector 334 can be secured to the barrel 306 by the interlocking face 340 and flange 324. Accordingly, the connector assembly 312 can enable the injector body 302 to generate and withstand very high pressures.

The configuration of the connector assembly 312 can be modified in many different ways. For example, although the connector assembly 312 is depicted as including three discrete connectors 334-338, in other embodiments, the connector assembly 312 can include fewer connectors (e.g., the first connector 334 can be integrated with the second connector 336, the second connector 336 can be integrated with the third connector 338) or more connectors (e.g., any of the first, second, and/or third connectors 334-338 can be separated into additional discrete connectors). As another example, the threaded couplings between any of the first, second, and/or third connectors 334-338 can be replaced or combined with other types of connections, such as snap fit couplings, interference fit couplings, etc. Optionally, the connector assembly 312 can include a quick release mechanism that enables the first, second, and/or third connectors 334-338 to be rapidly disconnected from each other, which can be beneficial for disassembling the injector body 302 for cleaning and/or loading the injector body 302 with additional embolic composition.

FIG. 3I is a top view of the tip 304, FIG. 3J is a cross-sectional view of the tip 304, and FIG. 3K is a closeup cross-sectional view of a portion of the tip 304 shown in FIG. 3J. The tip 304 is configured to deliver the embolic composition from the injector body 302 into a conduit of the treatment system (e.g., the conduit of the delivery system 101 of FIGS. 1A-2D). As best seen in FIG. 3K, the tip 304 includes a body 360, a plurality of elongated shafts (e.g., a first elongated shaft 362, a second elongated shaft 364, a third elongated shaft 366, and/or a fourth elongated shaft 392), and an insert 368. Some or all of the elongated shafts can be concentric and/or otherwise overlap each other to provide additional mechanical strength to the tip 304 and to prevent the tip 304 from failing, e.g., due to high pressure. The insert 368 is positioned within the body 360 and can surround at least a portion of some of the elongated shafts (e.g., the first and second elongated shafts 362, 364) to secure the elongated shafts to the body 360.

As illustrated in FIGS. 3J and 3K, the body 360 of the tip 304 is a generally tubular (e.g., cylindrical or hexagonal) structure extending from a proximal end portion 370 to a distal end portion 372. The body 360 includes an internal lumen 374 (e.g., a cavity) that has a first opening 376 (e.g., an aperture) at the proximal end portion 370 and a second opening 378 (e.g., an aperture) at the distal end portion 372. In some embodiments, the internal lumen 374 narrows from the first opening 376 to the second opening 378 so that the first opening 376 is larger than the second opening 378. For example, the first opening 376 can have a diameter greater than or equal to 0.1 inches, 0.15 inches, 0.167 inches, 0.2 inches, or 0.25 inches, while the second opening 378 can have a diameter less than or equal to 0.05 inches, 0.04 inches, 0.03 inches, 0.02 inches, or 0.01 inches.

As shown in FIG. 3I, the proximal end portion 370 of the body 360 can include a coupling portion 382 configured to connect the tip 304 to the injector body 302 of FIGS. 3A-3C. For example, the coupling portion 382 can mate with a corresponding coupling portion 326 (FIG. 3A) at the distal end portion 322 of the barrel 306. In some embodiments, the coupling portion 382 of the tip 304 is a female Luer lock, and the coupling portion 326 of the barrel 306 is a male Luer lock, or vice-versa. A Luer lock connection can be advantageous for withstanding the high pressures described herein. Alternatively or in combination, other types of coupling mechanisms can be used (e.g., snap fit, interference fit, fasteners, etc.).

The first elongated shaft 362 fluidly couples the tip 304 and injector body 302 to the conduit of the treatment system. As best seen in FIG. 3J, the first elongated shaft 362 is a hollow elongated structure having a lumen 388 extending from a proximal end portion 384 to a distal end portion 386. The lumen 388 can be fluidly coupled with the internal lumen 374 of the body 360 to form a passageway for the embolic composition. The first elongated shaft 362 can include a portion that is disposed within and coupled to the body 360, and a portion that is disposed outside of the body 360. For example, as best seen in FIG. 3K, the proximal end portion 384 of the first elongated shaft 362 is positioned within the internal lumen 374. The first elongated shaft 362 can extend through the second opening 378 of the body 360, such that the distal end portion 386 is located distal to the distal end portion 372 of the body 360 (FIG. 3J). In some embodiments, the first elongated shaft 362 is coupled to the body 360 at or near the second opening 378 (e.g., by welding, adhesives, interference fit, etc.). The first elongated shaft 362 can have a diameter that is less than or equal to 0.05 inches, 0.04 inches, 0.03 inches, 0.02 inches, or 0.01 inches, and/or the lumen 388 can have a diameter less than or equal to 0.02 inches, 0.015 inches, 0.014 inches, 0.013 inches, 0.012 inches, 0.011 inches, or 0.01 inches.

The second elongated shaft 364 is positioned within the internal lumen 374 of the body 360 to secure the first elongated shaft 362 within the body 360. As shown in FIG. 3K, the second elongated shaft 364 is a hollow elongated structure that surrounds (e.g., is concentric with) the portion of the first elongated shaft 362 located in the internal lumen 374 of the body 360. In some embodiments, the second elongated shaft 364 is coupled (e.g., welded) to the outer surface of the first elongated shaft 362. The second elongated shaft 364 can have a length less than or equal to 0.05 inches, 0.04 inches, 0.035 inches, 0.03 inches, or 0.02 inches. Optionally, the proximal end of the second elongated shaft 364 can be positioned distal to the proximal end portion 384 of the first elongated shaft 362. For example, the proximal end of the second elongated shaft 364 can be spaced apart from the proximal end portion 384 of the first elongated shaft 362 by a distance of at least 0.005 inches, 0.01 inches, 0.015 inches, 0.02 inches, or 0.025 inches, e.g., to prevent the second elongated shaft 364 from blocking the lumen 388 of the first elongated shaft 362. Alternatively, the proximal end of the second elongated shaft 364 can be aligned with or be positioned proximal to the proximal end portion 384 of the first elongated shaft 362.

In some embodiments, the second elongated shaft 364 is configured to abut a portion of the body 360. For example, the inner wall of the distal end portion 372 of the body 360 can include a stop 380 within the internal lumen 374 surrounding the second opening 378. The second opening 378 can be sized so that the first elongated shaft 362 can extend past the stop 380 and through the second opening 378, while the distal end of the second elongated shaft 364 is wider than the second opening 378 and contacts the stop 380. Because the first elongated shaft 362 is coupled to the second elongated shaft 364, the contact between the second elongated shaft 364 and the stop 380 can prevent the first elongated shaft 362 from being pushed through the second opening 378 because of the internal forces (e.g., high pressures) exerted on the first elongated shaft 362. In other embodiments, however, the second elongated shaft 364 is optional and can be omitted from the tip 304.

The insert 368 can secure the first and second elongated shafts 362, 364 within the body 360. As illustrated in FIG. 3K, the insert 368 can be a structure that conforms to and fills at least a portion of the internal lumen 374 of the body 360, including the spaces between the first and second elongated shafts 362, 364 and the inner wall of the body 360. The insert 368 can include an aperture extending therethrough that is sized to accommodate the first and second elongated shafts 362, 364. Accordingly, the insert 368 can surround and couple to the first and/or second elongated shafts 362, 364 to secure these components within the body 360 and provide additional mechanical strength. For example, the insert 368 can be fixed to the outer surfaces of the first and/or second elongated shafts 362, 364, e.g., via adhesives, interference fit, or other attachment techniques. In some embodiments, the insert 368 includes a curable material (e.g., a UV activated adhesive), which solidifies when cured or otherwise activated to bond the first and second elongated shafts 362, 364 in place within the internal lumen 374. Optionally, the insert 368 can be integrally formed with the body 360.

In the illustrated embodiment, the insert 368 extends proximally from the stop 380 to a location distal to the proximal end portion 370 of the body 360. In other embodiments, however, the insert 368 can be flush with the proximal end portion 370 of the body 360. As shown in FIG. 3J, the insert 368 includes a proximal face 390 that is concave and/or angled so that any embolic composition that is pressed against the proximal face 390 is directed into the lumen 388 of the first elongated shaft 362. In other embodiments, the proximal face 390 can be a flat surface.

The third elongated shaft 366 can serve as a strain reliever that reduces strain and/or other forces applied externally to the tip 304 (e.g., to the first elongated shaft 362). In some embodiments, the third elongated shaft 366 is a hollow elongated structure that is coupled to and surrounds (e.g., is concentric with) a portion of the first elongated shaft 362 to provide mechanical support and reinforcement. For example, the third elongated shaft 366 can be welded to the outer surface of the first elongate shaft 362. The proximal end of the third elongated shaft 366 can be disposed within the second opening 378 of the body 360 and can be coupled to the inner wall of the body 360. The third elongated shaft 366 can extend distally past the distal end portion 372 of the body 360, with the distal end of the third elongated shaft 366 located proximal to the distal end portion 386 of the first elongated shaft 362. In the illustrated embodiment, the third elongated shaft 366 is positioned distal to the second elongated shaft 364, with the proximal end of the third elongated shaft 366 spaced apart from the distal end of the second elongated shaft 364, such that the second and third elongated shafts 364, 366 surround different portions of the first elongated shaft 362 and do not overlap. The third elongated shaft 366 can have a diameter of at least 0.01 inches, 0.02 inches, 0.025 inches, 0.03 inches, or 0.04 inches, and a length of at least 0.5 inches, 1 inch, 1.5 inches, 2 inches, or 3 inches. Alternatively, the third elongated shaft 366 is optional and can be omitted from the tip 304.

In some embodiments, the tip 304 includes a fourth elongated shaft 392 that supports and/or protects the first and third elongated shafts 362, 366. The fourth elongated shaft 392 can be a hollow elongated structure that couples to and surrounds (e.g., is concentric with) the portions of the first and third elongated shafts 362, 366 that are external to the body 360 of the tip 304. In the illustrated embodiment, the proximal end of the fourth elongated shaft 392 abuts the distal end portion 372 of the body 360. In other embodiments, the proximal end of the fourth elongated shaft 392 can be disposed within the second opening 378 of the body 360 and coupled to the inner wall of the body 360. The distal end of the fourth elongated shaft 392 can be located proximal to the distal end of the third elongated shaft 366 and/or the distal end portion 386 of the first elongated shaft 362. The fourth elongated shaft 392 can have a length of at least 1 inch, 1.5 inches, 2 inches, 2.5 inches, 3 inches, or 4 inches. In some embodiments, the fourth elongated shaft 392 is made of shrink tubing or another polymeric material. Alternatively, the fourth elongated shaft 392 is optional and can be omitted from the tip 304.

The configuration of the tip 304 illustrated in FIGS. 3I-3K can be modified in many different ways. For example, although the tip 304 is depicted as including four elongated shafts, in other embodiments, the tip 304 can include fewer elongated shafts (e.g., any of the second, third, and/or fourth elongated shafts 364, 366, 392 can be omitted) or more elongated shafts. Additionally, the dimensions (e.g., length, diameter) of any of the elongated shafts can be varied as desired. The couplings between any of the elongated shafts can be achieved using welding, bonding, adhesives, interference fit, fasteners, and/or any other suitable attachment techniques.

The injector device 300 can include one or more components that are made from high strength materials, such as metallic materials. For example, any of the components of the injector body 302 (e.g., the barrel 306, rod 308, connector assembly 312, body 360, etc.) and/or the tip 304 (e.g., the body 360, first elongated shaft 362, second elongated shaft 364, third elongated shaft 366, fourth elongated shaft 392, etc.) can be formed from a metallic material, such as stainless steel and/or Nitinol. The use of metallic materials can ensure the injector device 300 maintains its structural integrity during operation, since other materials (e.g., plastics) may begin to act as a liquid instead of a solid or otherwise fail at very high pressures.

As previously noted, the injector device 300 can be used to introduce an embolic composition into a treatment system (e.g., the delivery system 101 of FIGS. 1A-2D). To use the injector device 300, an embolic composition is first loaded into the barrel 306 of the injector body 302 by inserting the embolic composition through the first opening 316 of the barrel 306 and into the inner chamber 314. The rod 308 can then be inserted into the barrel 306 and secured via the connector assembly 312. The tip 304 can be coupled to the distal end portion 322 of the barrel 306 before or after loading the embolic composition and inserting the rod 308.

During use, an operator can connect the injector device 300 to the treatment system by inserting the tip 304 into a port of the treatment system (e.g., the port 110 of the delivery system 101 shown in FIG. 1A). In some embodiments, the distal end portion 386 of the first elongated shaft 362 is inserted into the port, thereby forming a passageway from the injector body 302, through the tip 304, and into the conduit of the treatment system. The operator can manipulate the rod 308 via the handle 310 so that the rod 308 pressurizes the embolic composition. For example, the operator can rotate the handle 310, which causes the rod 308 to translate distally within the barrel 306 and compress the embolic composition with the pushing member 332. The operator can continue to rotate the handle 310 to increase the pressure (e.g., by further translating the rod 308 distally within the barrel 306) until the pressure is sufficiently high to push the embolic composition out through the second opening 320 of the barrel 306, through the internal lumen 374 of the tip 304 and the lumen 388 of the first elongated shaft 362, through the conduit of the treatment system, and into the treatment site (e.g., an aneurysm cavity).

In some embodiments, the injector device 300 generates at least 4,000 psi of pressure. For example, the injector device 300 can pressurize the embolic composition to at least 4,000 psi, 5,000 psi, 6,000 psi, 7,000 psi, 8,000 psi, 9,000 psi, 10,000 psi, 11,000 psi, 12,000 psi, 13,000 psi, 14,000 psi, 15,000 psi, or higher. These pressures can enable the injector device 300 to deliver the embolic composition to a treatment site at a sufficiently high flow rate for treatment purposes, such as a rate of at least 0.05 mL/minute, 0.1 mL/minute, 0.15 mL/minute, 0.2 mL/minute, 0.25 mL/minute, 0.3 mL/minute, 0.35 mL/minute, 0.4 mL/minute, 0.45 mL/minute, 0.5 mL/minute, or more. As discussed above, the injector device 300 can include several features that enable the injector device 300 to handle these high pressures without failing. For example, the connector assembly 312 can prevent the rod 308 from moving rearward due to high pressure when the operator releases the handle 310. Additionally, the use of multiple overlapping elongated shafts (e.g., the first, second, third, and fourth elongated shafts 362, 364, 366, 392) can reduce the likelihood of the tip 304 experiencing mechanical failure due to high pressure. Accordingly, the injector device 300 can be used to deliver the highly viscous embolic compositions described herein.

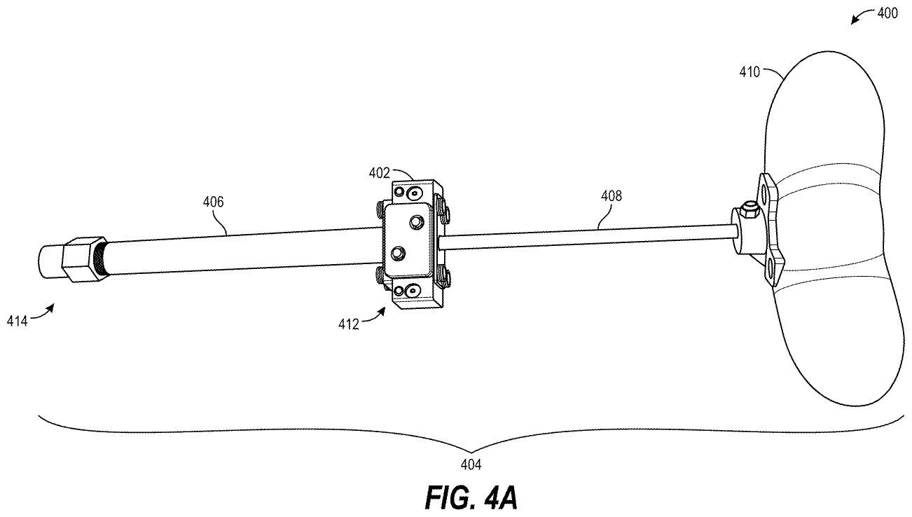

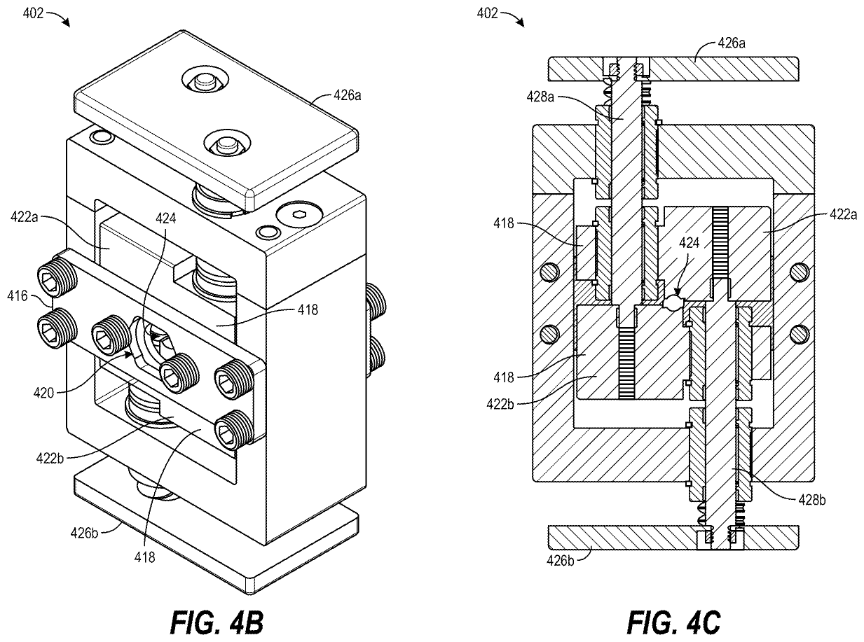

FIG. 4A is a perspective view of an injector device 400 including a pressure release mechanism 402 configured in accordance with embodiments of the present technology. The injector device 400 can be generally similar to the injector device 300 of FIGS. 3A-3K. For example, as shown in FIG. 4A, the injector device 400 includes an injector body 404 configured to couple to a tip (e.g., the tip 304 of the injector device 300 of FIGS. 3A-3K) for delivering an embolic composition into a treatment system (e.g., the system 100 of FIGS. 1A-1D). The injector body 404 includes a barrel 406 configured to hold an embolic composition, a rod 408 that is movable within the barrel 406 to pressurize the embolic composition, and a handle 410 that is actuatable by an operator to move the rod 408 within the barrel 406 to control the delivery of the embolic composition. Accordingly, the following discussion of the injector device 400 will be limited to those features that differ from the injector device 300 of FIGS. 3A-3K.

The pressure release mechanism 402 is configured to mitigate issues with overfilling that may arise when delivering a highly pressurized embolic composition into an aneurysm. In some instances, when the operator stops actuating the handle 410, there may be residual pressure within the barrel 406 that continues pushing the embolic composition out of the injector device 400 and into the aneurysm cavity, thus causing injection of a larger volume of the embolic composition than intended. This may lead to overfilling of the aneurysm and leakage of the embolic composition into the parent vessel, which can have catastrophic consequences for the patient (e.g., emboli formation and stroke). To overcome this issue, the pressure release mechanism 402 can be configured to rapidly dissipate the residual pressure within the injector body 404 to stop the injection of the embolic composition and prevent overfilling.

In some embodiments, the pressure release mechanism 402 maintains or dissipates the pressure within the injector body 404 by controlling the position of the rod 408 relative to the barrel 406. As shown in FIG. 4A, the barrel 406 of the injector body 404 is an elongated, hollow structure (e.g., a generally cylindrical tube) extending between a proximal end portion 412 and a distal end portion 414. The pressure release mechanism 402 can be coupled to the proximal end portion 412 of the barrel 406, and also to the portion of the rod 408 proximal to the barrel 406. The pressure release mechanism 402 can be movable between a first configuration in which the pressure release mechanism 402 prevents the rod 408 from moving proximally relative to the barrel 406, thus maintaining the pressure within the barrel 406; and a second configuration in which the pressure release mechanism 402 permits the rod 408 to move proximally relative to the barrel 406, thus allowing the pressure within the barrel 406 to dissipate by displacing the rod 408 backwards from the barrel 406.

FIGS. 4B and 4C are perspective and front cross-sectional views, respectively, of the pressure release mechanism 402. The pressure release mechanism 402 includes a first connector 416 configured to couple to the barrel 406, and a second connector 418 configured to couple to the rod 408. The first connector 416 can be a bracket, plate, etc., defining a first aperture 420 shaped to receive the proximal end portion 412 of the aperture. As best seen in FIG. 4C, the second connector 418 can include an upper connector portion 422a (e.g., an upper block) and lower connector portion 422b (e.g., a lower block) that collectively define a second aperture 424 shaped to receive the rod 408. The upper connector portion 422a and lower connector portion 422b can be threaded along the periphery of the second aperture 424 to mate with the threading on the rod 408. In some embodiments, the threading is located along only a portion of the periphery of the second aperture 424, e.g., the curved upper and lower portions of the second aperture 424 shown in FIG. 4C can be threaded, while the remaining portions of the second aperture 424 are unthreaded. This configuration permits the rod 408 to be disengaged from the second aperture 424 when the upper connector portion 422a and lower connector portion 422b are separated from each other, as described below.

To maintain the pressure within the barrel 406 (e.g., when injecting the embolic composition), the pressure release mechanism 402 can be placed in a first configuration (shown in FIGS. 4B and 4C) in which the upper connector portion 422a and lower connector portion 422b are positioned in close proximity to each other, such that the second aperture 424 has a first, smaller size and the threading around the second aperture 424 engages the threading on the rod 408. Accordingly, the pressure release mechanism 402 can prevent the rod 408 from moving proximally relative to the barrel 406 so that the pressure exerted on the embolic composition by the rod 408 is sustained.

To release the pressure within the barrel 406 (e.g., when terminating the injection of the embolic composition), the operator can push an upper button 426a and/or a lower button 426b located on the upper and lower sides of the pressure release mechanism 402, respectively. The upper button 426a can be coupled to the lower connector portion 422b via an upper shaft 428a, and the lower button 426b can be coupled to the upper connector portion 422a via a lower shaft 428b. When the upper button 426a is pushed, the upper shaft 428a can translate downward to move the lower connector portion 422b downward and away from the upper connector portion 422a. Similarly, when the lower button 426b is pushed, the lower shaft 428b can translate upward to move the upper connector portion 422a upward and away from the lower connector portion 422b. Accordingly, the pressure release mechanism 402 can be transitioned into a second configuration in which the upper connector portion 422a and lower connector portion 422b are spaced apart from each other, such that the second aperture 424 has a second, larger size and the threading around the second aperture 424 is disengaged from the threading on the rod 408. Thus, the rod 408 can move proximally relative to the pressure release mechanism 402 and the barrel 406 to dissipate residual pressure within the barrel 406. In some embodiments, the upper button 426a and/or lower button 426b are spring-loaded so the pressure release mechanism 402 is biased toward the first configuration, thus avoiding inadvertent depressurization when injecting the embolic composition.

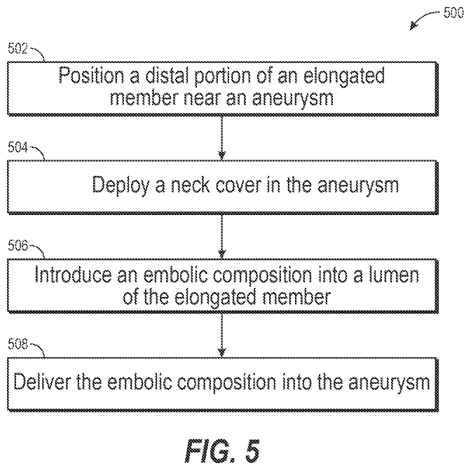

FIG. 5 is a flow diagram illustrating an example method 500 of treating an aneurysm in accordance with embodiments of the present technology. The method 500 can be performed by any embodiment of the systems and devices described herein, such as by a treatment system including an injector device. For examples, any of the steps of the method 500 can be performed with the treatment system 100 described in FIGS. 1A-2D, the injector device 300 of FIGS. 3A-3K, and/or the injector device 400 of FIGS. 4A-4C.

At block 502, the method 500 begins with positioning a distal portion of an elongated member near the aneurysm. The elongated member can be a part of the treatment system that can be inserted into a patient and intravascularly advanced to a desired location. For example, the elongated member can be the third elongated shaft 108 of the delivery system 101 of FIGS. 1A-2D. The distal portion of the elongated member can be advanced through the patient until the distal portion is positioned adjacent, near, or within the aneurysm.

At block 504, the method 500 optionally includes deploying a neck cover in the aneurysm. As previously described with respect to FIGS. 1A-2D, the neck cover (e.g., neck cover 120) can be coupled to the distal portion of the elongated member. The neck cover can initially be in a low-profile state (e.g., contained within another elongated member, such as the second elongated shaft 106). The elongated member can be advanced through the patient until the neck cover is positioned within the aneurysm. The operator can then deploy the neck cover by transitioning the neck cover from the low-profile state to an expanded state, e.g., by releasing the neck cover from the other elongated member so the neck cover self-expands into the expanded state. Once deployed within the aneurysm, the neck cover can generally conform to the shape of the aneurysm sac, as previously described with respect to FIG. 2A. In other embodiments, however, the neck cover is optional and block 502 can be omitted.

At block 506, the method 500 can continue with introducing the embolic composition into a lumen of the elongated member. As discussed above, this process can include coupling an injector device (e.g., the injector device 300 of FIGS. 3A-3K or the injector device 400 of FIGS. 4A-4C) loaded with the embolic composition to the treatment system. When the embolic composition is loaded into the injector device, the embolic composition can already have the desired viscosity for treating an aneurysm, such that the viscosity does not substantially increase during delivery into the aneurysm. For example, the embolic composition can have a viscosity (e.g., dynamic viscosity measured at 20° C.) of at least 50 Pa-s, 100 Pa-s, 200 Pa-s, 500 Pa-s, or 1000 Pa-s before and/or during loading into the injector device. Alternatively or in combination, the embolic composition can have a storage modulus (e.g., measured within the linear viscoelastic region at 37° C.) of at least 50 Pa, 80 Pa, 100 Pa, 200 Pa, 300 Pa, 400 Pa, 500 Pa, or 600 Pa before and/or during loading into the injector device. After the injector device is loaded with the embolic composition, the injector device can then be coupled to the treatment system to form a passageway between the injector device and the lumen of the elongated member. To introduce the embolic composition into the treatment system, the injector device can pressurize the embolic composition to a relatively high pressure (e.g., at least 10,000 psi), e.g., as described above with respect to the injector device 300 of FIGS. 3A-3K.

At block 508 the method 500 can proceed with delivering the embolic composition into the aneurysm, e.g., as previously described with respect to FIG. 2B. The pressure generated from the injector device can provide a sufficient force to deliver the embolic composition through the lumen of the elongated member to the distal portion of the elongated member and into the aneurysm. The delivery can be complete when the embolic composition fills a desired amount of the aneurysm sac.

In embodiments where a neck cover is used, as the embolic composition fills the aneurysm, it can press downward on the neck cover to compress or otherwise deform the neck cover to a lower volume state, e.g., as previously described with respect to FIG. 2C. This can stabilize the neck cover over the aneurysm neck and can also be used to monitor the extent of filing of the aneurysm cavity. In some embodiments, the method 400 optionally includes detaching the neck cover from the elongated member after the embolic composition has been delivered to the aneurysm, e.g., as previously described with respect to FIG. 2D.

### CONCLUSION

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

To the extent any materials incorporated herein by reference conflict with the present disclosure, the present disclosure controls.

Unless otherwise indicated, all numbers expressing dimensions, percentages, or other numerical values used in the specification and claims, are to be understood as being modified in all instances by the term “about.” Accordingly, unless indicated to the contrary, the numerical parameters set forth in the specification and attached claims are approximations that may vary depending upon the desired properties sought to be obtained by the present technology. At the very least, and not as an attempt to limit the application of the doctrine of equivalents to the scope of the claims, each numerical parameter should at least be construed in light of the number of reported significant digits and by applying ordinary rounding techniques. Additionally, all ranges disclosed herein are to be understood to encompass any and all subranges subsumed therein. For example, a range of “1 to 10” includes any and all subranges between (and including) the minimum value of 1 and the maximum value of 10, i.e., any and all subranges having a minimum value of equal to or greater than 1 and a maximum value of equal to or less than 10, e.g., 5.5 to 10.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1-51. (canceled)

52. A method of treating an aneurysm, comprising:
positioning a distal portion of an elongated member near or within an aneurysm;
introducing an embolic composition into a lumen of the elongated member using an injector device coupled to a proximal portion of the elongated member, wherein the injector device is configured to pressurize the embolic composition to a pressure of at least 10,000 psi; and
delivering the embolic composition into the aneurysm via the elongated member.

53. The method of claim 52, wherein the embolic composition comprises a storage modulus of at least 80 Pa at 37° C. within a linear viscoelastic range of the embolic composition.

54. The method of claim 53, further comprising loading the embolic composition into the injector device, wherein the embolic composition comprises the storage modulus of at least 80 Pa at 37° C. within the linear viscoelastic range of the embolic composition before being loaded into the injector device.

55. The method of claim 52, wherein the embolic composition is delivered into the aneurysm at a rate of at least 0.2 milliliters per minute.

56. The method of claim 52, wherein the injector device comprises an injector body coupled to a tip.

57. The method of claim 56, wherein the injector body and the tip are made at least partially out of a metallic material.

58. The method of claim 56, wherein the tip comprises a body having a distal opening, a proximal opening, and an internal lumen extending between the distal and proximal openings.

59. The method of claim 58, wherein at least a portion of the distal opening has a diameter of less than 0.025 inches.

60. The method of claim 58, wherein the tip comprises:
a first elongated shaft disposed within the internal lumen of the body and extending through the distal opening, the first elongated shaft having an outer surface; and
a second elongated shaft disposed within the internal lumen of the body, wherein the second elongated shaft is fixed to the outer surface of the first elongated shaft and surrounds a first portion of the first elongated shaft, and wherein the second elongated shaft is wider than the distal opening.

61. The method of claim 60, wherein the tip comprises an insert disposed within the internal lumen of the body around the first and second elongated shafts.

62. The method of claim 60, wherein the tip comprises a third elongated shaft coupled to the outer surface of the first elongated shaft so that the third elongated shaft surrounds a second portion of the first elongated shaft that is spaced apart from the first portion.

63. The method of claim 52, further comprising deploying a neck cover from the elongated member while the distal portion of the elongated member is positioned near or within the aneurysm such that the neck cover self-expands to assume a first expanded state within the aneurysm.

64. The method of claim 63, wherein delivering the embolic composition into the aneurysm causes the neck cover to transform into a second expanded state, the second expanded state having a smaller interior volume than the first expanded state.

65. The method of claim 52, further comprising:
terminating delivery of the embolic composition into the aneurysm, and
dissipating residual pressure within the injector device using a pressure release mechanism.

66. A method of treating an aneurysm, the method comprising:
positioning a distal end of an elongated shaft in an aneurysm cavity;
introducing an embolic composition into a lumen of the elongated shaft using an injector device, wherein the injector device is coupled to a proximal portion of the elongated shaft; and
delivering an embolic composition into the aneurysm cavity, wherein the embolic composition has a storage modulus of at least 80 Pa at 37° C. within a linear viscoelastic range of the embolic composition, and wherein the embolic composition is delivered into the aneurysm cavity at a rate of at least 0.2 milliliters per minute.

67. The method of claim 66, wherein delivering the embolic composition comprises pressurizing the embolic composition to a pressure of at least 10,000 psi.

68. The method of claim 66, wherein the injector device includes a tip, and wherein the tip comprises a body having a distal aperture.

69. The method of claim 68, wherein the tip comprises:
a first elongated shaft having an outer surface, the first elongated shaft being disposed at least partially within the body and extending through the distal aperture; and
a second elongated shaft disposed within the body and fixed to the outer surface of the first elongated shaft such that the first elongated shaft is received within the second elongated shaft along a portion of its length, wherein the second elongated shaft is wider than the distal aperture.

70. The method of claim 66, further comprising loading the embolic composition into the injector device, wherein the embolic composition comprises the storage modulus of at least 80 Pa at 37° C. within the linear viscoelastic range of the embolic composition before being loaded into the injector device.

71. The method of claim 66, further comprising:
terminating delivery of the embolic composition into the aneurysm, and
dissipating residual pressure within the injector device using a pressure release mechanism.
